(19) United States
(12) Patent Application Publication
(10) Pub. No.: US 2025/0127657 A1
(43) Pub. Date: Apr. 24, 2025

(54) VAPORIZED OPHTHALMIC DRUG DELIVERY DEVICE

(71) Applicant: Verily Life Sciences LLC, Dallas, TX (US)

(72) Inventors: Maxim Armstrong, South San Francisco, CA (US); Allister McGuire, South San Francisco, CA (US)

(21) Appl. No.: 18/797,334

(22) Filed: Aug. 7, 2024

Related U.S. Application Data
(60) Provisional application No. 63/532,081, filed on Aug. 11, 2023.

(51) Int. Cl. A61F 9/00

(52) U.S. Cl. CPC A61F 9/0008

(57) ABSTRACT

An eye dropper with sensors that allows for delivery of a specific or controlled dose of a therapeutic agent in vapor form to a patient's eye is provided.

This nonprovisional patent application claims the benefit of the earlier filing date of U.S. provisional application No. 63/532,081 filed 11 Aug. 2023.

### TECHNICAL FIELD

The present disclosure relates to an eye dropper with sensors that allows for delivery of a specific or controlled dose of a therapeutic agent in vapor form to a patient's eye.

### BACKGROUND

When using an eye dropper that administers a drug in liquid form, it is often difficult to deliver the drug to the user's tear film. With passive eye drops, it is common that the user blinks before the drop reaches the eye, blinks shortly after the drop reaches the eye thereby ejecting most of the drop, or misses the eye altogether. Similar issues with blinking and aim exist in some powered eye drop and spray devices. Therefore, there is a need for an ophthalmic drug delivery device that accurately delivers a controlled dose of an ophthalmic drug to a patient's tear film.

### SUMMARY

In an aspect, a therapeutic agent delivery device is provided. The device can include a vapor chamber configured to be positioned about an eye of a patient to deliver a therapeutic agent in vapor form to a tear film of a patient's eye. The vapor chamber can comprise a therapeutic agent concentration sensor and a camera. A therapeutic agent source can be included that comprises a therapeutic agent in communication with the vapor chamber. A heater and/or vacuum can be in communication with the vapor chamber and the therapeutic agent source. The heater and/or vacuum can be configured to convert the therapeutic agent in the therapeutic agent source into a vapor form. A controller compartment can be operably coupled to the vapor chamber and can comprise a timer and a microcontroller comprising a non-transitory memory having computer-executable instructions stored thereon and a processor to access the memory and execute the instructions to at least receive a signal from the camera, receive a signal from the timer, receive a signal from the therapeutic agent concentration sensor, determine the surface area of the patient's tear film that is exposed to the therapeutic agent based on the camera signal, determine the length of time the patient's tear film is exposed to the therapeutic agent based on the timer signal, and determine the concentration of the therapeutic agent in vapor form in the vapor chamber based on the therapeutic agent sensor.

### BRIEF DESCRIPTION OF THE DRAWINGS

FIG. 1 is a perspective view of a therapeutic agent delivery device according to an aspect of the present disclosure.

FIG. 2 is a side schematic view of the therapeutic agent delivery device FIG. 1 sealed against a user's face about the surface of the user's eye.

FIG. 3 is a schematic illustration of the components of a therapeutic agent delivery device according to an aspect of the present disclosure.

FIG. 4 is a schematic illustration of the components of a therapeutic agent delivery device according to an aspect of the present disclosure.

FIG. 5 is a block diagram of components of a therapeutic agent delivery device according to an aspect of the present disclosure.

FIG. 6 is a block diagram of components of a therapeutic agent delivery device according to an aspect of the present disclosure.

FIG. 7 is a block diagram of components of a microcontroller of a therapeutic agent delivery device according to an aspect of the present disclosure.

FIG. 8 is a flow diagram depicting steps of a method of using or method of operation of a therapeutic agent delivery device according to an aspect of the present disclosure.

FIG. 9 is a flow diagram depicting steps of a method of using or method of operation of a therapeutic agent delivery device according to an aspect of the present disclosure.

FIG. 10 is a flow diagram depicting steps of a method of using or method of operation of a therapeutic agent delivery device according to an aspect of the present disclosure.

### DETAILED DESCRIPTION

As used herein with respect to a described element, the terms “a,” “an,” and “the” include at least one or more of the described element(s) including combinations thereof unless otherwise indicated. Further the term “a,” “an,” and “the” can refer to one component performing a described functionality or more than one component performing the same functionality. Further, the terms “or” and “and” refer to “and/or” and combinations thereof unless otherwise indicated. Along the same lines, by “substantially” is meant that the shape or configuration of the described element need not have the mathematically exact described shape or configuration of the described element but can have a shape or configuration that is recognizable by one skilled in the art as generally or approximately having the described shape or configuration of the described element. As such “substantially” refers to the complete or nearly complete extent of a characteristic, property, state, or structure. The exact allowable degree of deviation from the characteristic, property, state, or structure will be so as to have the same overall result as if the absolute characteristic, property, state, or structure were obtained. A component operably coupled or connected to another component can have intervening components between the components so long as the device can perform the stated purpose. The term “therapeutic agent” and “drug” are used interchangeably herein. A processor or microcontroller as described herein can include a single processor and/or microcontroller or multiple processors and/or microcontroller that perform the disclosed steps or execute the disclosed instructions.

Although the drawings show certain elements of a device in combination, it should be noted that such elements can be included (or excluded) in other embodiments or aspects illustrated in other drawings or otherwise disclosed in the specification. In other words, each of the disclosed aspects and embodiments of the present disclosure may be considered individually or in combination with other aspects and embodiments of the disclosure including patent applications incorporated by reference herein.

The present disclosure relates to therapeutic agent delivery devices to deliver an accurate and controlled dose of a therapeutic agent in vapor form to the patient's eye. One of the limiting factors of delivering ophthalmic drugs via eye drops is that the dose can vary dramatically among individuals and drop-to-drop. This dose variability can affect the efficacy of treatment and the risk of side effects. Indeed, some drugs may be ruled out as eye drops because of a small tolerance for dose variability. Devices that seek to deliver ophthalmic drugs to the tear film via a vapor suffer similar challenges with dose variability. Therapeutic agent delivery devices disclosed herein address these challenges by delivering a therapeutic agent in vapor form and incorporating sensors that measure key factors for delivered drug dose and provide feedback to ensure an effective and safe dose is delivered.

In general, therapeutic agent delivery devices as disclosed herein provide a temporary chamber formed between the user's face and the device, thereby allowing a predictable dose of therapeutic agent to dissolve at the air-tear interface of the eye. In particular, the devices deliver a controlled dose of an ophthalmic drug through the use of a suite of sensors that measure, for example, the surface area of the tear film that is exposed to a therapeutic agent, the amount of time the tear film is exposed to a therapeutic agent, and the concentration of the vaporized drug or droplets. The device may predict the total dose of drug delivered using the relationship described by Equation 1, for example. Based on this sensed information, the device can calculate how much therapeutic agent is delivered to the patient's tear film.

$${n}_{DRUG} = {\int }_{0}^{t} {A}_{TF} \cdot {J}_{DRUG} ( {C}_{VAPOR} , {C}_{TF} ) dt \qquad ( 1 )$$

where:
n_{DRUG} is the total dose of drug delivered to the tear film, A_{TF} is the exposed area of the tear film, J_{DRUG} is the flux of drug from vapor to the tear film, C_{VAPOR} is the concentration of drug in vapor, C_{TF} is the concentration of drug in the tear film, and t is time

Using the solubility of the drug in the tear film and the drug's diffusion constant (both which will be characterized ahead of time), the total drug (i.e. dose) that has been delivered to the eye can be calculated since the drug concentration of the delivered vapor and the duration that concentration was exposed to which area of the tear film is known.

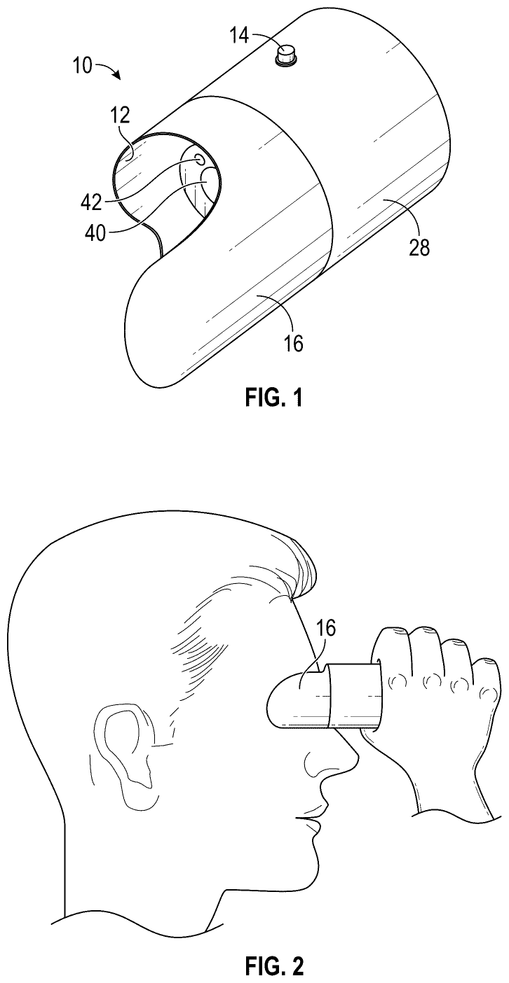

In an embodiment and with reference to FIGS. 1 and 2, a therapeutic agent delivery device 10 can comprise, for example, a hand-held device that can be held up to a user's eye to deliver a therapeutic agent to a tear film of a user's eye. The device can comprise an elastomeric seal 12 that can allow the user to form an enclosed chamber between his or her eye and the device. Into this chamber, the device can vaporize a known amount of liquid (or other form) of drug stored in a therapeutic agent source. A button 14 or other user input component can be used to initiate drug dispensation. The amount of drug that will dissolve in the tear film can be dictated by the incorporation of different sensors (as described in more detail below) that determine, for example, drug vaporized concentration/quantity and duration and surface area of tear film exposure.

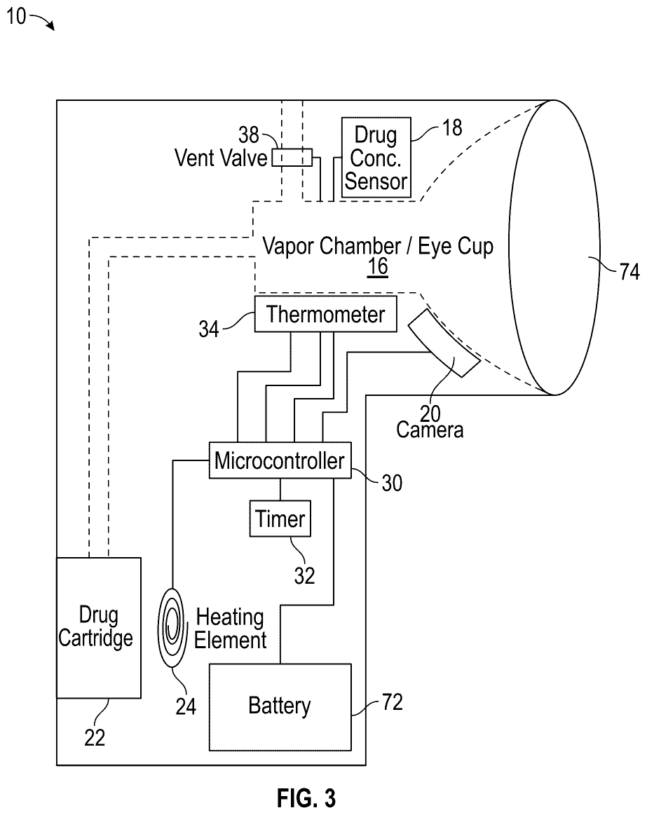

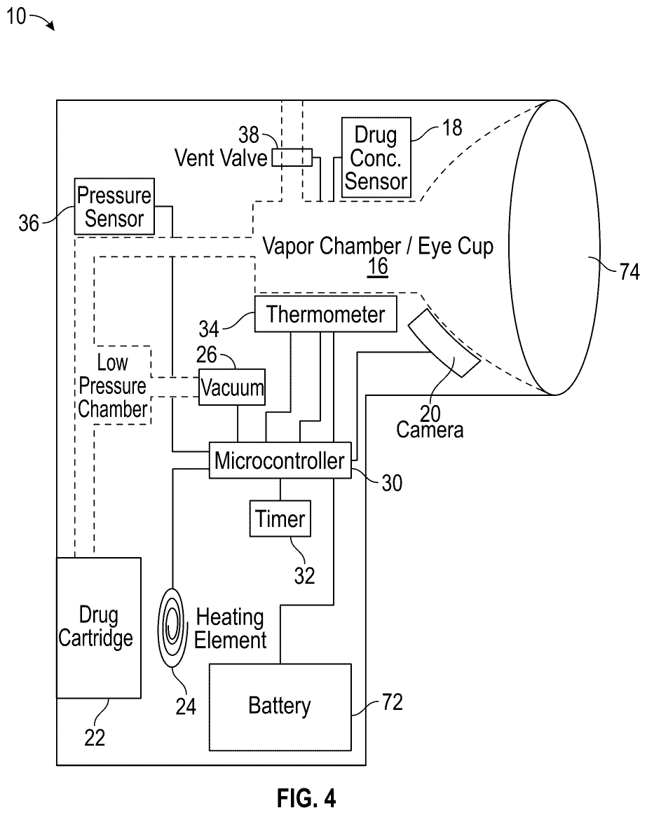

Referring to FIGS. 1-6, therapeutic agent delivery device 10 can comprise vapor chamber 16 configured to be positioned about an eye of a patient to form a sealed chamber against the patient's eye. As depicted in FIGS. 3-4, the sealed chamber can effectively be in the shape of an “eye cup” 74. Vapor chamber 16 can comprise therapeutic agent concentration sensor 18 and camera 20. Therapeutic agent concentration sensor 18 can be used to determine the concentration of therapeutic agent in vapor form in the vapor chamber. Non-limiting examples of therapeutic agent concentration sensors include a spectrometer to obtain real-time information to calculate how much cumulative drug is in the vapor chamber, an ammeter measuring the current delivered to the heater 24, to inferentially determine how much energy was dissipated to sublimate or evaporate the therapeutic agent with a known enthalpy of vaporization, or other suitable drug concentration sensors. Camera 20 can be used to measure the surface area of the tear film of the user's eye that is exposed. The camera can also be used to monitor the seal of the vapor chamber against the user's eye to ensure that therapeutic agent does not escape from the vapor chamber such that some amount of therapeutic agent delivered to the user is inefficacious. In other words, the camera can be used to determine the amount of therapeutic agent that does not escape from the vapor chamber, rendering the remaining concentration in the vapor chamber too low to deliver an effective dose to the tear film. Therapeutic agent source 22, which can be, for example, a drug reservoir or a single or multi-day drug cartridge can be in communication with vapor chamber 16. The device can include orifice 40 through which vapor is emitted into the vapor chamber. Heater 24 and/or vacuum 26 can be in communication with vapor chamber 16 and therapeutic agent source 22 and can be configured to convert the therapeutic agent (which can be, for example, in liquid, semi-solid, or solid form) in therapeutic agent source 22 into a vapor form. The heater can be a hot plate, for example, which is within vapor chamber 16 or elsewhere in the device (e.g., behind the vapor chamber) so long as the heater is in communication with the therapeutic agent source and the vapor chamber. In the case of a vacuum, the vacuum can comprise, for example, a small piston and cylinder controlled by a motor or solenoid. The vacuum can be in a separate chamber or compartment such that the vaporized drug is released into the vapor chamber against the user's face at ambient pressure. In certain instances, the device can comprise both a heater and a vacuum to allow, for example, drug to vaporize faster and use less heat compared to vapor generated by a heater alone.

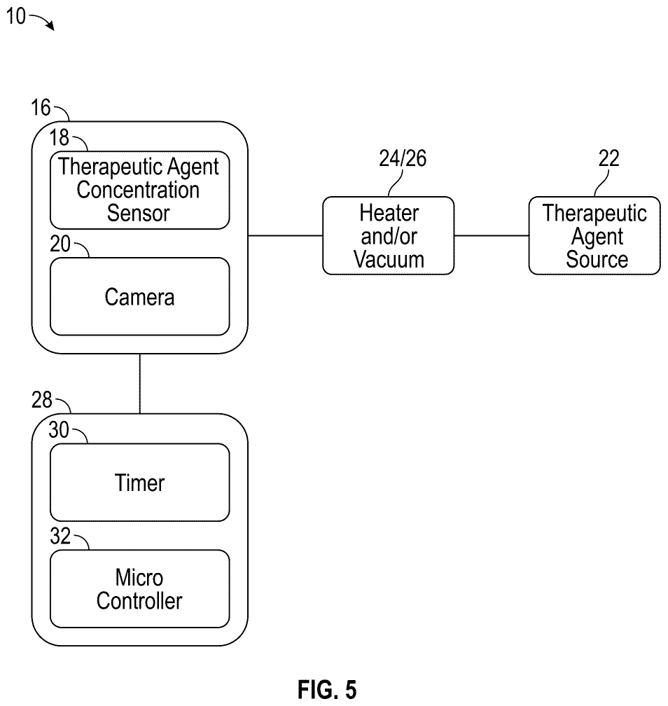

Referring to FIGS. 1, 3 and 4, the device can further include controller component 28 operably coupled to vapor chamber 16. Controller component 28 can comprise microcontroller 30 and timer 32. Although FIGS. 3 and 4 illustrate the timer as being a separate component from the microcontroller, the timer can be on the same circuit board as the microcontroller or can be located elsewhere on or in the device. The timer can be used to determine the duration for which the user's tear film is exposed. Referring to FIG. 5, microcontroller 30 can comprise processor 34 and non-transitory memory 36. Memory 36 can include computer-readable instructions that, when executed by processor 34, cause the microcontroller to perform various functions attributed throughout this disclosure to the microcontroller. The computer-readable instructions can be encoded within memory 36. The memory can comprise non-transitory computer-readable storage media including any suitable volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media with the sole exception being a transitory, propagating signal. The processor can include one or more conventional processors, microprocessors, or specialized dedicated processors that include processing circuitry operative to interpret and execute computer readable program instructions, such as program instructions for controlling the operation and performance of one or more of the various other components of the device for implementing the functionality, steps, and/or performance of the present embodiments.

In particular, the memory can include computer-executable instructions 38 stored thereon that, when executed by processor 34 cause microcontroller 30 to at least receive a signal from the camera, receive a signal from the timer, receive a signal from the therapeutic agent concentration sensor, determine the surface area of the patient's tear film that is exposed based on the camera signal, determine the length of time the patient's tear film is exposed based on the timer signal, and determine the concentration of the therapeutic agent in vapor form in the vapor chamber based on the therapeutic agent concentration sensor. Based on these determinations, the microcontroller can direct delivery of a desired/pre-determined dose range of therapeutic agent to the patient's tear film.

The controller component can also include other components such as, for example, other electronic components such as a battery 72 and charging port. The controller component can also include a button to start the delivery cycle, a buzzer/beeper/light emitting diode 42 to indicate the status of drug delivery or to assist the user in aligning the device with the user's eye.

The device can comprise other sensors as well and thus the microcontroller can comprise computer-executable instructions to receive other sensor signals and determine parameters of drug delivery based on these sensor signals. For example, and with reference to FIGS. 3 and 4, the vapor chamber of the device can include temperature sensor 34, such as a thermometer, and/or pressure sensor 36. When a heater is utilized, the temperature sensor can measure the temperature of the vapor in the vapor chamber to generate a temperature signal. Computer-executable instructions stored in the memory of the microcontroller, when executed by the processor, can cause the microcontroller to receive the temperature signal and modulate the heat generated by the heater based on the temperature signal to modify the concentration of therapeutic agent delivered through the vapor chamber and/or modify the vapor temperature for the comfort of the user. When a vacuum is utilized, the vacuum chamber can include pressure sensor 36 to measure the pressure within the vacuum chamber to generate a pressure signal. Computer-executable instructions stored in the memory of the microcontroller, when executed by the processor, can cause the microcontroller to receive the pressure signal and modulate the pressure created by the vacuum based on the pressure sensor signal to modify the concentration of therapeutic agent delivered through the vapor chamber.

The vapor chamber can also include vent valve 38 that allows therapeutic agent to escape from the vapor chamber once a therapeutically sufficient/desired/pre-determined amount or dose range of drug or otherwise suitable amount of drug has been delivered from the vapor chamber. To this end, computer-executable instructions stored in the memory of the microcontroller, when executed by the processor, can cause the microcontroller to actuate the valve to cause drug release once an appropriate/desired amount of drug has been delivered from the vapor chamber. The actuation of the valve can be based on the camera signal, the timer signal, the therapeutic agent concentration signal, the temperature signal, the pressure signal, or combinations thereof.

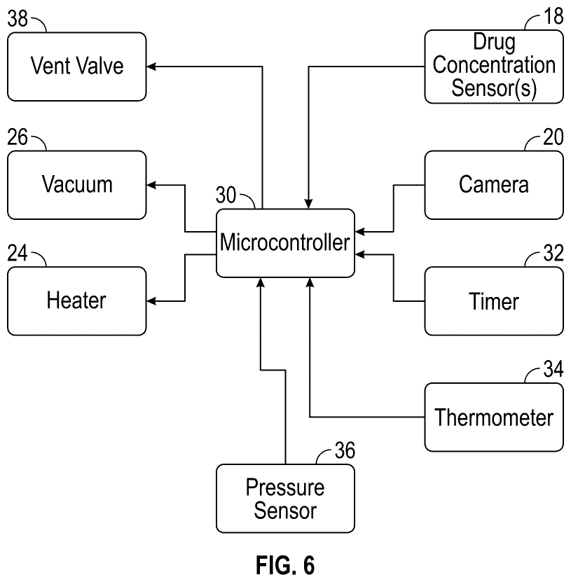

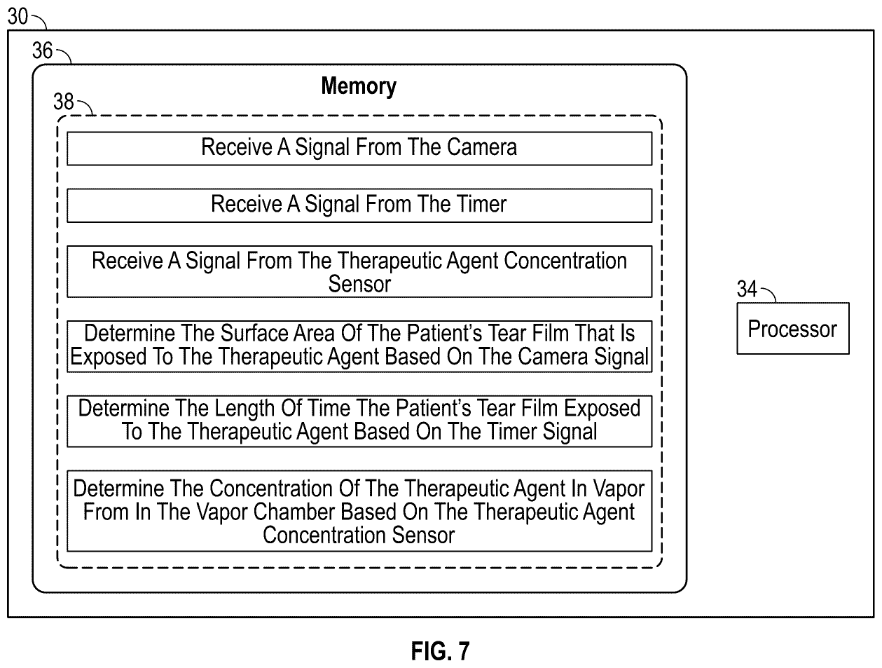

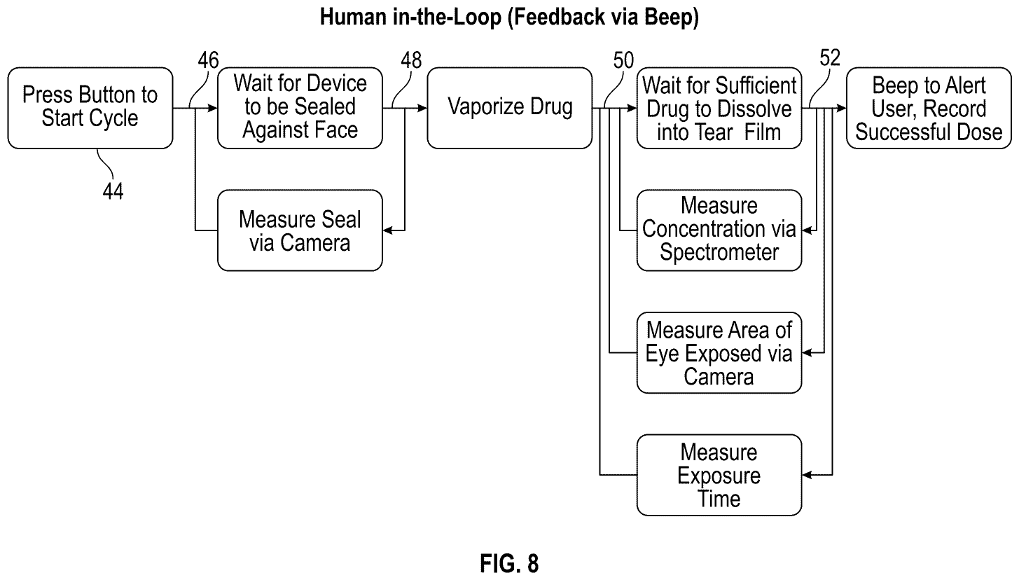

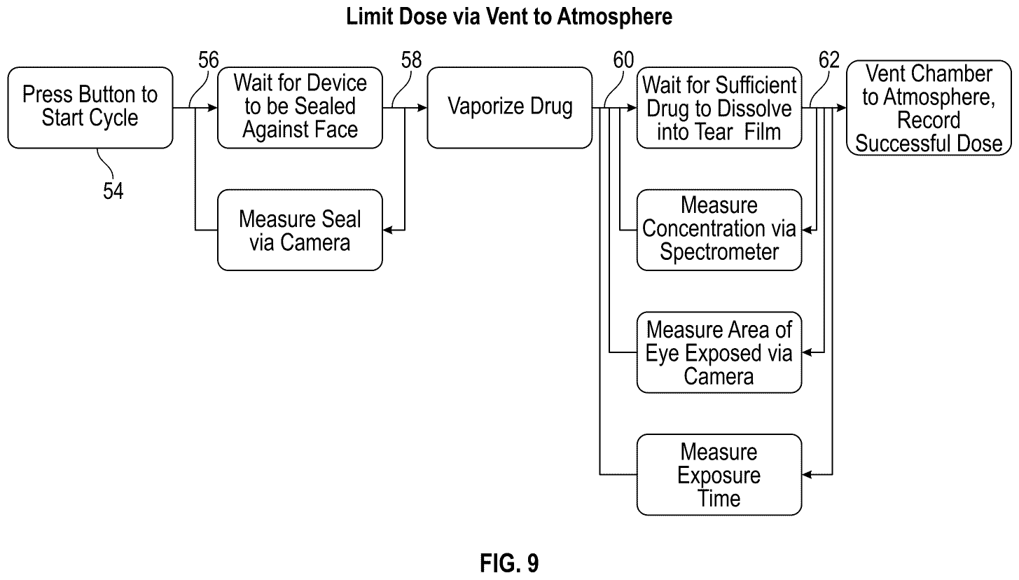

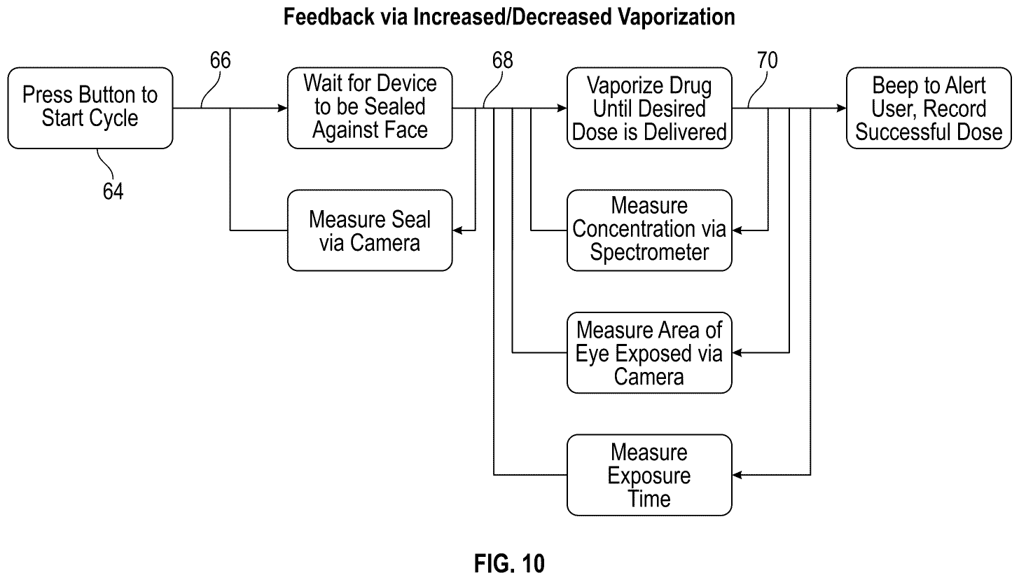

FIGS. 8-10 provide exemplary flow diagrams of diverse ways of determining that a successful/pre-determined dose of therapeutic agent has been delivered to the user. Referring to FIG. 8, at step 44, a user can place the device against his or her face and press a button on the device to initiate the therapeutic agent delivery process. At step 46, the user can wait for the device to be sealed against his or her face in the region about the user's eye. Once a proper seal has been determined via the camera in the device, a processor can execute instructions to vaporize the therapeutic agent at step 48. At step 50, a concentration of the therapeutic agent can be measured via e.g. a spectrometer, the area of the patient's eye that is exposed can be measured by a camera, and the time the user's eye is exposed can be measured by a timer. Such sensed parameters can be used to determine when a sufficient/pre-determined dose range of drug has dissolved into the user's tear film. Once this determination has been made, at step 52, a processor can execute instructions to cause the device to beep or to otherwise alert the user that a successful dose has been delivered and such delivery can also be recorded by the processor. Upon receiving this alert, the user can withdraw the device from his or her face. With respect to FIG. 9, steps 54 to 60 can be similar to steps 44 to 50 of FIG. 6, except at step 62, the processor can execute instructions to vent the vapor chamber to the atmosphere to release any excess drug above the pre-determined dose range. Such a step removes the user's involvement with respect to withdrawing the device upon receiving a notification indicating that a sufficient dose of therapeutic agent has been delivered thereby preventing excess drug from being delivered. Rather, in the steps outlined in FIG. 9, the device will automatically release excess drug via the vent. In FIG. 10, the device can rely on increased or decreased vaporization of the therapeutic agent to control the dose of drug delivered. Steps 64 and 66 can be similar to steps 44 and 46 of FIG. 8 and steps 54 and 56 of FIG. 9. At step 68, the therapeutic agent can be vaporized until a desired dose is delivered by measuring the concentration of the therapeutic agent, the area of the eye exposed, and the exposure time. For example, the heat generated by the heater and/or the pressure generated by the vacuum can be modulated to control the degree of drug vaporization. Once a sufficient/predetermined dose range of therapeutic agent is delivered, a processor can execute instructions to cause the device to beep or to otherwise alert the user that a successful dose has been delivered and such delivery can also be recorded by the processor at step 70.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into (or excluded from) other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. A therapeutic agent delivery device comprising:
a vapor chamber configured to be positioned about an eye of a patient to deliver a therapeutic agent in vapor form to a tear film of a patient's eye, the vapor chamber comprising:
a therapeutic agent concentration sensor; and
a camera;
a therapeutic agent source comprising a therapeutic agent in communication with the vapor chamber;
a heater and/or vacuum in communication with the vapor chamber and the therapeutic agent source, the heater and/or vacuum configured to convert the therapeutic agent in the therapeutic agent source into a vapor form; and
a controller compartment operably coupled to the vapor chamber comprising:
a timer; and
a microcontroller comprising a non-transitory memory having computer-executable instructions stored thereon and a processor to access the memory and execute the instructions to at least:
receive a signal from the camera;
receive a signal from the timer;
receive a signal from the therapeutic agent concentration sensor;
determine the surface area of the patient's tear film that is exposed to the therapeutic agent based on the camera signal;
determine the length of time the patient's tear film is exposed to the therapeutic agent based on the timer signal; and
determine the concentration of the therapeutic agent in vapor form in the vapor chamber based on the therapeutic agent sensor signal.

2. The therapeutic agent delivery device of claim 1, wherein the vapor chamber further comprises an elastomeric seal to form an enclosed chamber between the patient's eye and the vapor chamber.

3. The therapeutic agent delivery device of claim 1, wherein the therapeutic agent delivery device is a handheld device.

4. The therapeutic agent delivery device of claim 1, wherein the therapeutic agent source comprises:
a reservoir comprising a therapeutic agent or a cartridge comprising a therapeutic agent.

5. The therapeutic agent delivery device of claim 1, wherein the therapeutic agent in the therapeutic agent source is in solid, semi-solid, or liquid form.

6. The therapeutic agent delivery device of claim 1 further comprising a pressure sensor, temperature sensor, a vent valve, or combinations thereof contained within the vapor chamber.

7. The therapeutic agent delivery device of claim 1, wherein the microcontroller and the timer are integrated on a circuit board.

8. The therapeutic delivery device of claim 1, further comprising a battery, a charging part, button to start drug delivery, a buzzer/beeper/light emitting diode to indicate status, or combinations thereof contained on or within the controller compartment.

9. The therapeutic agent delivery device of claim 1, wherein the therapeutic agent concentration sensor is a spectrometer or an ammeter.

10. The therapeutic agent delivery device of claim 1, further comprising a temperature sensor and/or a pressure sensor.

11. The therapeutic agent delivery device of claim 10, wherein the temperature sensor is configured to measure the temperature of vapor in the vapor chamber to generate a temperature signal.

12. The therapeutic agent delivery device of claim 11, further comprising computer-executable instructions stored in the memory that, when executed by the processor, cause the microcontroller to:
receive the temperature signal; and
modulate the heat generated by the heater based on the temperature signal to modify the concentration of therapeutic agent delivered through the vapor chamber and/or modify the vapor temperature for the comfort of the user.

13. The therapeutic agent delivery device of claim 10, wherein the pressure sensor is configured to measure the pressure within the vacuum chamber to generate a pressure signal.

14. The therapeutic agent delivery device of claim 13, further comprising computer-executable instructions stored in the memory that, when executed by the processor, cause the microcontroller to:
receive the pressure signal; and
modulate the pressure created by the vacuum based on the pressure signal to modify the concentration of therapeutic agent delivered through the vapor chamber.

15. The therapeutic agent delivery device of claim 1, further comprising computer-executable instructions stored in the memory that, when executed by the processor, cause the microcontroller to:
receive a signal from the camera; and
determine the amount of therapeutic agent that does not escape from the vapor chamber based on the camera signal.

16. The therapeutic agent delivery device of claim 1, further comprising:
a vent valve in the vapor chamber; and
computer-executable instructions stored in the memory that, when executed by the processor, cause the microcontroller to:
receive the camera signal, the timer signal, the therapeutic agent concentration signal, a temperature signal, a pressure signal, or combinations thereof; and
actuate the vent valve to allow the therapeutic agent to release from the vapor chamber once a desired amount of therapeutic agent has been delivered, the actuation based on the camera signal, the timer signal, the therapeutic agent concentration signal, the temperature signal, the pressure signal, or combinations thereof.
